(19) United States
(12) Patent Application Publication
(10) Pub. No.: US 2017/0296673 A1
(43) Pub. Date: Oct. 19, 2017

(54) CONSERVATION OF BIOACTIVITY BY HYDROPHOBIC MATRICES

(71) Applicants: Andreas VOIGT, Berlin (DE); Sonja LEHMANN, Berlin (DE); Anja VOIGT, Berlin (DE); Therakine BioDelivery GmbH, Berlin (DE)

(72) Inventors: Andreas VOIGT, Berlin (DE); Sonja LEHMANN, Berlin (DE); Anja VOIGT, Berlin (DE)

(21) Appl. No.: 15/516,687

(22) PCT Filed: Oct. 6, 2015
(86) PCT No.: PCT/US15/54229

§ 371 Date: Apr. 4, 2017

Related U.S. Application Data
(60) Provisional application No. 62/060,654, filed on Oct. 7, 2014.

(51) Int. Cl. A61K 47/44; A61K 47/22; A61K 47/12; A61K 47/42; A61K 36/064

(52) U.S. Cl. CPC A61K 47/44; A61K 47/42; A61K 36/064; A61K 47/12; A61K 47/22

(57) ABSTRACT

The present invention discloses a drug delivery system comprising at least one hydrophobic matrix and at least one pharmaceutically active compound. The hydrophobic matrix comprises at least one hydrophobic solid component and at least one hydrophobic liquid component. The pharmaceutically active compound may comprise a biological cell or a biopolymer. The hydrophobic matrix conserves the activity of and protects the functionality of the biological cell or biopolymer in hostile environment, at elevated temperature, cold temperatures or a combination thereof. The present invention also discloses the use of the drug delivery system in a kit for diagnostic or analytical purposes. Additionally, the present invention discloses the use of the drug delivery system to deliver the pharmaceutically active compound to a subject who has or is suspected of having a pathophysiological condition.

### CROSS-REFERENCE TO RELATED APPLICATIONS

The present application is a national stage entry according to 35 U.S.C. §371 of PCT Application No. PCT/US2015/054229 filed on Oct. 6, 2015, which claims priority to U.S. Provisional Application Ser. No. 62/060,654 filed on Oct. 7, 2014.

### TECHNICAL FIELD

The subject matter herein generally relates to the field of controlled drug release. Specifically, drug delivery compositions are disclosed comprising cell-wall containing cells and large class of biopolymers and hydrophobic matrix, methods to manufacture such compositions and the use of these drug delivery composition. More importantly, the bioactivity of these cell-wall containing cells and large class of biopolymers may be conserved when contained in a hydrophobic environment.

### BACKGROUND

Most therapeutic dosage forms include mixtures of one or more pharmaceutically active compounds with additional components referred to as excipients. The pharmaceutica1ly active compounds include substances that are used in the prevention, treatment, or cure of a disease. The pharmaceutically active compounds can be naturally occurring or synthetic substances, or can be produced by recombinant methods, or any combination of these approaches.

Numerous methods have been devised for delivering these pharmaceutically active compounds into living organisms with more or less success. Traditional oral therapeutic dosage forms include both solids (for example, tablets, capsules, pills, etc.) and liquids (for example, solutions, suspensions, emulsions, etc.). Parenteral dosage forms include solids and liquids as well as aerosols (administered using inhalers, etc.), injectables (administered using syringes, micro-needle arrays, etc.), topicals (for example, foams, ointments, etc.), and suppositories, among other dosage forms. Although these dosage forms might be effective in delivering low molecular weight active ingredients, each of these various methods suffers from one or more drawbacks, including the lack of bioavailability as well as the inability to completely control either the spatial or the temporal component of the pharmaceutically active compound's distribution when it comes to high molecular weight pharmaceutically active compounds. These drawbacks are especially challenging for administering biotherapeutics, i.e. pharmaceutically active peptides (e.g. growth factors), proteins (e.g. enzymes, antibodies), oligonucleotides and nucleic acids (e.g. RNA, DNA, PNA, aptamers, spiegelmers), hormones and other natural substances or synthetic substances mimicking such, since many types of pharmacologically active biomolecules are at least partially broken down, either in the digestive tract or in the blood system, and delivered sub-optimally to the target site.

Therefore, there is an ongoing significant need for improved drug-delivery methods in the life sciences, including, but not limited to, human and veterinary medicine. One important goal for any new drug-delivery method is to deliver the desired therapeutic agent(s) to a specific site in the body over a specific and controllable period of time, i.e. controlling the delivery of one or more substances to specific organs and tissues in the body in both a spatial and temporal manner.

While delivering the desired therapeutic agent(s), especially the ones comprising at least one biological cell to a specific site in the body over a specific and controllable period of time, the therapeutic agent(s) usually must pass through hostile environments that may adversely interact with the therapeutic agent(s). For example, the environment can be incompatible with the therapeutic agents due to the hydrophobic or hydrophilic nature of the environment, have high temperature, have low pH, be poisonous, or exhibit other similar detrimental environmental conditions. In a hostile environment, the desired therapeutic agent(s) may lose bioactivity due to denaturation or degradation. As such, the bioactivity must sometimes be conserved in a hydrophobic matrix. However, hydrophobic environments may reduce or destroy the bioactivity of the therapeutic agent(s), especially the ones comprising at least one biological cell.

Methods for accomplishing this spatially and temporally controlled delivery are known as controlled-release drug-delivery methods. Delivering pharmaceutically active ingredients to specific organs and tissues in the body offers several potential advantages, including increased patient efficacy, extending activity, lowering the dosage required to reach the intended target site, minimizing detrimental side effects, and permitting the use of more potent therapeutics. In some cases, controlled-release drug-delivery methods can even allow the administration of therapeutic agents which would otherwise be too toxic or ineffective for use.

There are five broad types of solid dosage forms for controlled-delivery oral administration: reservoir and matrix diffusive dissolution, osmotic, ion-exchange resins, and prodrugs. For parenterals, most of the above solid dosage forms are available as well as injections (intravenous, intramuscular, etc.), transdermal systems, and implants. Numerous products have been developed for both oral and parenteral administration, including depots, pumps, and micro- and nano-particles.

The incorporation of active ingredients into polymer matrices acting as a core reservoir is one approach for controlling their delivery. Contemporary approaches for formulating such drug delivery systems are dependent on technological capabilities as well as the specific requirements of the application. The following are two main structural approaches for sustained delivery systems: the release controlled by diffusion through a barrier such as shell, coat, or membrane, and the release controlled by the intrinsic local binding strength of the pharmaceutically active ingredient(s) to the core or to other ingredients in the core reservoir.

Another strategy for controlled delivery of therapeutic agents, especially for delivering biotherapeutics, involves their incorporation into polymeric micro- and nano- particles either by covalent or cleavable linkage or by trapping or adsorption inside porous network structures. Various particle architectures can be obtained, for instance core/shell structures. Typically one or more pharmaceutically active ingredients are contained either in the core, in the shell, or in both components. Their concentration can be different throughout the respective component in order to modify the pattern. However, their small size allows them to diffuse in and out of the target tissue or being successfully attacked by macrophages. The use of intravenous nano-particles is further limited due to rapid clearance by the reticuloendothelial system. Porosity also allows organic solvents to enter and limit or destroy the bioactivity of the active ingredient. Notwithstanding this, polymeric microspheres remain an important delivery vehicle.

There is a significant unmet need for a delivery system that allows the delivery of therapeutic agents comprising at least one biological cell or biopolymer. Specifically, there is a significant unmet need for a delivery system that conserves the activity of the biological cell or biopolymer and efficiently delivers the biological cell or biopolymer to the intended target site.

### SUMMARY

Various embodiments are described herein, and do not limit the scope in any way.

In a non-limiting embodiment, a drug delivery composition is described. In another embodiment, this drug delivery system comprises at least one hydrophobic matrix, and at least one pharmaceutically active compound. In yet another embodiment, the hydrophobic matrix comprises at least one hydrophobic solid component and at least one hydrophobic liquid component. In still yet another embodiment, the hydrophobic solid component and the hydrophobic liquid component of the hydrophobic matrix have a stronger binding affinity with each other than with the pharmaceutically active compound. In another embodiment, the hydrophobic solid component comprises an anti-caking agent, the anti-caking agent is a compound selected from the group consisting of magnesium stearate, magnesium palmitate and similar compounds.

In yet another embodiment, the hydrophobic solid component is selected from the group consisting of waxes, fruit wax, carnauba wax, bees wax, waxy alcohols, plant waxes, soybean waxes, synthetic waxes, triglycerides, lipids, long-chain fatty acids and their salts like magnesium stearate, magnesium palmitate, esters of long-chain fatty acids, long-chain alcohols like cetyl palmitate, waxy alcohols, long-chain alcohols like cetyl alcohol, oxethylated plant oils, oxethylated fatty alcohols. In still yet another embodiment, the hydrophobic liquid component acts as a glue to bind the hydrophobic solid component together. In another embodiment, the hydrophobic liquid component is selected from the group consisting of plant oils, castor oil, jojoba oil, soybean oil, silicon oils, paraffin oils, and mineral oils, cremophor, oxethylated plant oils, and oxethylated fatty alcohols. In yet another embodiment, the hydrophobic liquid component is labeled with at least one agent selected from the group consisting of small molecules, hormones, peptides, proteins, phospholipids, polysaccharides, mucins and biocompatible polymers. In still yet another embodiment, the biocompatible polymers comprise polyethylene glycol (PEG), dextran or another similar material.

In another embodiment, the pharmaceutically active compound is either alone or in combination with at least one excipient. In yet another embodiment, the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. In yet another embodiment, the pharmaceutically active compound is selected from the group consisting of a living organelle, a cell, a tissue constituent, a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof. In still yet another embodiment, the living organelle, the cell or the tissue constituent has a cell wall, where the cell wall protects the bioactivity of the pharmaceutically active compound from hydrophobic properties of the hydrophobic matrix.

In another embodiment, the hydrophobic matrix and the pharmaceutically active compound are in a paste-like or a semi-solid form. In yet another embodiment, the pharmaceutically active compound is dispersed in the hydrophobic matrix in a particulate form, in a microparticulate form or in a dissolved state. In still yet another embodiment, the pharmaceutically active compound is dissolved in an aqueous solution. In another embodiment, the aqueous solution comprises water, electrolytes, sugars, or low and high molecular weight, water soluble passive ingredients. In yet another embodiment, the hydrophobic matrix comprises an aqueous solution. In still yet another embodiment, the aqueous solution comprises water, sugars, surfactant, buffer salts, stabilizers, amino acids, or low molecular weight carbohydrates. In another embodiment, the hydrophobic matrix is labeled with at least one agent selected from the group consisting of dyes, fluorophores, chemiluminescent agent, isotopes, metal atoms or clusters, radionuclides, enzymes, antibodies and tight binding partners, said tight binding partners comprising biotin or avidin.

In yet another embodiment, the hydrophobic matrix conserves activity of said pharmaceutically active compound in a hydrophobic environment, protects functionality of the pharmaceutically active compound in a hostile condition, hydrophobic environment or a combination thereof, provides stability to the pharmaceutically active compound at ambient or elevated temperatures, protects the pharmaceutically active compound from water-soluble poisonous substances, biological attack or a combination thereof, provides a temporary replacement for a cold chain, maintains bioactivity of the pharmaceutically active compound at higher temperature or a combination thereof.

In another embodiment, there is a kit comprising the above-mentioned drug delivery composition. In yet another embodiment, the drug delivery system in the kit allows detection, localization or imaging in a cell or a subject. In still yet another embodiment, the subject is an individual or an animal. In another embodiment, there is a method of treating a subject having or suspected of having a disease, comprising the above-mentioned drug composition. In still yet another embodiment, the disease is cancer, a bacterial infection, a viral infection, a parasitic infection, an inflammation, an immunological disease, a diabetes-related disease, a geriatric disease, or a metabolic disease. In yet another embodiment, the drug delivery system is administered orally, topically, intradermally, intranasally, intravenously, intraperitoneally, intracranially, intramuscularly, intravitreally or directly into a target site. In still yet another embodiment, the subject is a human or an animal.

### BRIEF DESCRIPTION OF THE DRAWINGS

FIGS. 1A-1B show results of the assays performed to examine stabilization of yeast cell at pH 1-stomach conditions where the yeast with hydrophobic matrix protection were compared with the yeast without protection in pH 1 at body temperature for 90 minutes. FIG. 1A shows that yeast cells with hydrophobic matrix survived based on the levels of carbon dioxide detected. FIG. 1B shows that yeast cells without protection (hydrophobic matrix) did not survive because no carbon dioxide was detected.

### DETAILED DESCRIPTION

The following language and descriptions of various embodiments are provided in order to further an understanding thereof. However, it will be understood that no limitations of the present embodiments are intended, and that further alterations, modifications, and applications of the principles of the present embodiments are also included.

As used herein, the term “hydrophobic matrix” refers to a material in which the pharmaceutically active compound is embedded. The hydrophobic solid component and the hydrophobic liquid component combine to form the matrix that enables the pharmaceutically active compound to be embedded within.

As used herein, the term “mass percent” is understood to refer to the mass of one component of the matrix, divided by the mass of the entire matrix, and multiply by 100%. For example, the mass percent of a pharmaceutically active compound may be determined by taking the mass of the pharmaceutically active compound within the matrix, dividing by the mass of the entire matrix, and multiplying by 100%. For instance, in one embodiment, the pharmaceutically active compound may be present at from about 0.1 mass percent to about 35 mass percent of the drug delivery system.

As used herein, the term “oil” refers to neutral, nonpolar chemical substance that is a viscous liquid at ambient temperatures. Some examples of oil that can be used in the different embodiments may include but are not limited to plant oil, castor oil, jojoba oil, soybean oil, cotton seed oil, olive oil, silicon oil, paraffin oil, and mineral oil, and oxethylated plant oil.

As used herein, the term “pharmaceutically active compound” refers to a compound or a combination of compounds that are used in manufacturing a drug product. This compound may also have a direct effect on the disease diagnosis, prevention, treatment or cure. Some examples of the pharmaceutically active compound that can be used herein are listed supra.

As used herein, the term “receptor antagonist” refers to a type receptor specific ligand or drug that can block receptor-mediated response by binding to the receptor and preventing the binding of agonists to the receptor. Some examples of such receptor antagonist include but are not limited to anti-TNF alpha, anti-Interleukin-1, anti-Interleukin-6, anti-epidermal growth factor receptor, anti-dopamine receptor, anti-Angiotensin II receptor, anti-aldosterone receptor and anti-leukotriene receptor.

As used herein, the term “anti-angiogenic compounds” refer to compounds that inhibit the growth of new blood vessels, reduce the production of pro-angiogenic factors, prevent the pro-angiogenic factors from binding to their receptors, and block the actions of pro-angiogenic factors or a combination thereof. Some examples of these compounds include but are not limited to compounds that inhibit the activity of VEGF, PDGF, and angiogenesis stimulators.

As used herein, the term “intracellular signaling inhibitors” refer to compounds that block signaling pathways by blocking the binding of ligands to the receptor involved in cell signaling or signal transduction, the actions of the receptors or the combination thereof. These compounds are useful in treatment, prevention, diagnosis or cure of various diseases. Some examples of intracellular signaling inhibitors include but are not limited to JAK1, JAK3 and SYK.

As used herein, the term “sustained release” refers to a dosage form designed to release a drug at a predetermined rate in order to maintain a constant drug concentration in the system for a specific period of time.

As used herein, the term “anti-caking agent” refers to an additive placed in powdered or granulated material to prevent the formation of lumps. Some examples of anti-caking agents include but are not limited to tricalcium phosphate, powdered cellulose, magnesium stearate, magnesium palmitate, sodium bicarbonate, sodium ferrocyanide, potassium ferrocyanide, calcium ferrocyanide, bone phosphate, sodium silicate, silicon dioxide, calcium silicate, magnesium trisilicate, talcum powder, sodium aluminosilicate, potassium aluminium silicate, calcium aluminosilicate, bentonite, aluminium silicate, stearic acid and polydimethylsiloxane.

As used herein, the term “microparticulate” refers to small, drug-containing low-molecular weight particles that are suspended in a liquid carrier medium.

As used herein, the term “tissue constituent” is intended to include, but is not limited to, any component of a tissue, for example any cellular component (e.g. cell membrane fraction, nuclear component, mitochondrial component, nucleotide, peptide, etc.).

As used herein, the term “excipient” is known in the art to refer to a natural or synthetic substance is formulated alongside the pharmaceutically active compound. There are several reasons for using the excipient in a drug composition because they act as a buffer, filler, binder, lubricant, or an osmotic agent. For instance, it may be used for the purpose of bulking up formulations that contain potent pharmaceutically active compounds. It may also be used to confer a therapeutic enhancement on the pharmaceutically active compound in the final dosage form, such as facilitating drug absorption or solubility. Further, it may also be used to assist in the handling of the pharmaceutically active compound by enabling powder consistency, non-stick properties or in vitro stability such as prevention of denaturation. Some of the factors that affect the selection of the excipient in a drug composition may include but is not limited to the route of administration, dosage form as well as the type of the pharmaceutically active compound in the drug composition. The various classes and types of pharmaceutically active compounds, excipients, polymers, and polyampholytes are familiar to those skilled in the art of drug delivery.

As used herein, the term “water soluble poisonous substance” refers to all kinds of biogenic substances, which are able to affect live cells or biologics, especially if they are incorporated into the drug delivery complex. Some examples of these substance include but are not limited to oxidizers, enzymes or antibodies.

As used herein, the term “cold chain” refers to a process of maintaining a storage temperature while the drug-delivery composition is transferred between places for storage.

As used herein, the term “intravitreally” refers to one of the routes of administration of a drug or other substance, wherein the drug or other substance is delivered into the vitreous, near the retina at the back of the eye. The vitreous is a jelly-like fluid that fills the inside of the eye.

The containment of biological cells or biopolymers in hydrophobic media is strongly reducing and even destroys their bioactivity or life functions. A drug delivery system may efficiently deliver pharmaceutically active compounds that include but are not limited to the list of the compounds listed supra. This drug delivery system may efficiently deliver cells, alternatively, cell wall containing cells such as bacteria like lactobacteria or yeast like Saccharomyces species) or biopolymers using a hydrophobic matrix. The drug delivery system comprises at least one hydrophobic matrix along with at least one pharmaceutically active compound. The hydrophobic matrix may comprise at least one hydrophobic solid component and at least one hydrophobic liquid component. The examples of the hydrophobic solid components and the hydrophobic liquid components is as discussed supra. In one embodiment, the hydrophobic solid component comprises an anti-caking agent, examples of which are provided supra. The hydrophobic liquid component may act as a glue to bind the hydrophobic solid component. In one embodiment, the hydrophobic solid component and the hydrophobic liquid components has a stronger binding affinity with each other than the pharmaceutically active compound. In another embodiment, the hydrophobic solid component may be conjugated with at least one agent selected from the group consisting of small molecules, hormones, peptides, proteins, phospholipids, polysaccharides, mucins and biocompatible polymers. Some examples of biocompatible polymers include but are not limited to polyethylene glycol (PEG), dextran or another similar material. This conjugation of the hydrophobic liquid component modifies its function, stability, rate of release of the pharmaceutically active compound or a combination thereof.

Some examples of pharmaceutically active compounds that can be delivered using this drug delivery system include but are not limited to the ones provided supra. In one embodiment, the living organelle, the cell or tissue constituent that can be delivered using this delivery system has a cell wall, where the cell wall protects the bioactivity of the pharmaceutically active compound from the hydrophobic properties of the hydrophobic matrix. Some examples of cell wall containing cells include but are not limited to bacteria like lactobacillus or yeasts like Saccharomyces species. Some examples of biopolymers that can be delivered using this drug delivery system may include but are not limited to therapeutic proteins, aptamers, carbohydrates or nucleic acids. In another embodiment, the pharmaceutically active compound may either be alone or in combination with at least one excipient. Excipients often may act as buffer, filler, binder, osmotic agent, lubricant or fulfill other similar functions. Polyampholytes are multiply charged polymers, which bear both anionic and cationic groups in the relevant medium, e.g. in an aqueous solution. The polyampholytes may fulfill all kinds of functions including but not limited to active drug (for example, a protein), passive ingredient in the interaction with an active drug or passive ingredient for drug release control (for example, swelling by water binding and helping to form channels for diffusion of actives out of the matrix). One skilled in the art of drug delivery is familiar and knowledgeable about the various types of pharmaceutically active compounds, excipients, polymers and polyampholytes that can be used in the drug delivery system.

In another embodiment, the pharmaceutically active compound may be dissolved in an aqueous solution. The aqueous solution comprises water, electrolytes, sugars or low and high molecular weight, water soluble passive ingredients. In yet another embodiment, the hydrophobic matrix and the pharmaceutically active compound are in a paste-like or semi-solid form. The hydrophobic matrix may comprise an aqueous solution, which comprises water, sugars, surfactants, buffer salts, stabilizers, amino acids, low and high molecular weight, carbohydrates. In another embodiment, the pharmaceutically active compound may be dispersed in the hydrophobic matrix in a particulate form, microparticulate form or in a dissolved state. In another embodiment, the hydrophobic matrix is labeled with at least one agent selected from the group consisting of dyes, fluorophores, chemiluminescent agent, isotopes, metal atoms or clusters, radionuclides, enzymes, antibodies and tight binding partners, said tight binding partners comprising biotin or avidin. This labeling allows this drug delivery system to be used to detect, locate or image or for any other analytical or medical purpose in a cell or subject.

The drug delivery system described herein has several applications. For instance, this drug delivery system can be used in a kit and used for medical or analytical purposes including but not limited to detection, localization or imaging in a cell or subject. The subject in this case can be a human or an animal. This drug delivery system may also be used to treat a subject having or suspected of having a disease, where the disease may be cancer, a bacterial infection, a viral infection, a parasitic infection, an inflammation, a diabetes-related disease, an immunological disease, a geriatric disease or a metabolic disease. The drug delivery system may be administered by several routes to this subject, including but not limited to oral, topical, intradermal, intranasal, intravenous, intraperitoneal, intracranial, intramuscular, intravitreal and directly into a target site. The subject may be a human or an animal.

The drug delivery system described herein is unique for several reasons. For instance, the drug delivery system conserves activity of the pharmaceutically active compound in a hydrophobic environment. Further, by embedding the pharmaceutically active compound within the hydrophobic matrix, the hydrophobic matrix protects the pharmaceutically active compound from harsh conditions in an aqueous environment such as low pH in the stomach of the subject. This is advantageous for probiotic, prebiotic or symbiotic applications of this drug delivery system, for example, lactobacillus and yeast in food) as allows for these cells to be active in hostile stomach environment. Another example is that the drug delivery system provides stability to the pharmaceutically active compound at ambient or elevated temperatures. This is particularly helpful in handling temperature-sensitive pharmaceutically active compounds such as vaccines or antibodies. Yet another example is that the drug delivery system protects the pharmaceutically active compound from water soluble poisonous substance such as oxidizers, enzyme poison or biological attack in an aqueous environment. Some examples of biological attack include but are not limited to oxidation, hydrolysis, cell death, immunological interaction or cell lysis. Another example is that the drug delivery system provides a temporary replacement for a difficult to establish cold chain where the drug delivery system is being transferred between places for storage. Further, the drug delivery system also maintains the bioactivity of the pharmaceutically active compound while being stored at higher temperature.

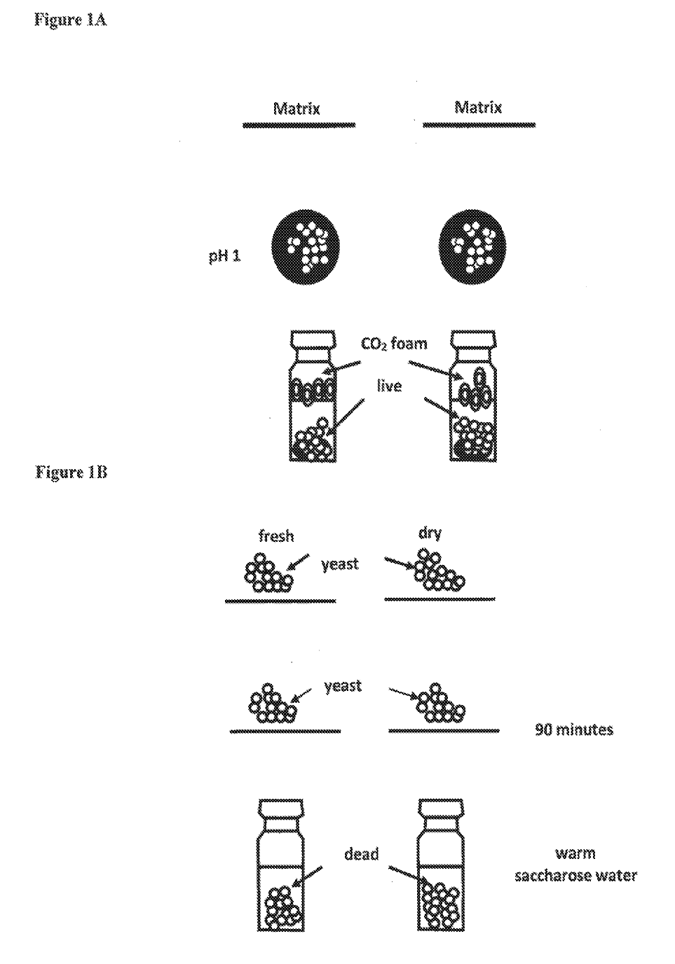

As an illustration, non-limiting examples are disclosed as to how the drug delivery system conserves the bioactivity of the pharmaceutically active compounds discussed herein, specifically, the biological cells or biopolymers. The conservation of the bioactivity of yeast embedded in the hydrophobic matrix was compared to unembedded yeast samples as discussed in Example 4. After destruction of the hydrophobic matrix, the yeast embedded within this matrix showed signs of survival based on the carbon dioxide detected (FIG. 1A). The unembedded yeast did not survive based on lack of detection of carbon dioxide (FIG. 1B).

### EXAMPLES

The following examples illustrate certain representative embodiments. It is to be understood that the following examples shall not limit the scope in any way.

### Example 1

Five grams of dry gelatin powder were added to 20 ml of raps oil. The mixture was then sealed and stored for 7 days at ambient temperature (about 22° C.). After 7 days, 75 ml of water was added to the mixture. The system was shaken for 5 minutes and stored for another 12 hours at ambient temperature. As a result, the whole system transformed into a gel, thereby demonstrating that the activity of the gelatin was not destroyed while stored in a hydrophobic environment.

### Example 2

Five grams of dry gelatin powder was added to 20 ml of raps oil. The mixture was then sealed and stored for 7 days at ambient temperature (about 22° C.). After the 7 days, 75 ml of water was added to the mixture. The system was shaken for 5 minutes and stored for another 12 hours at cool temperature (about 5° C.). It was observed that the whole system transformed into a gel, thereby demonstrating that the activity of the gelatin was not destroyed while stored in a hydrophobic environment.

### Example 3

Ten grams of dry (granulated) yeast of a Saccharomyces species was added to 20 ml of raps oil. The mixture was then sealed and stored for 5 days at ambient temperature. After the fifth day, 75 ml of saccharose-containing water at 40° C. was added and the system was shaken. The saccharose is just an example of the substrate for yeast metabolism, for example, 5 grams of saccharose in 75 ml of water. After a few minutes, carbon dioxide was detected, thereby showing that the bioactivity of the yeast was not destroyed while stored in a hydrophobic environment.

### Example 4

10 grams of fresh yeast of a Saccharomyces species was added to 20 ml of raps oil. The mixture was sealed and stored for 5 days at ambient temperature. After the fifth day, 75 ml of saccharose-containing water at 40° C. was added and the system was shaken. Carbon dioxide was detected after a few minutes, thereby showing that the bioactivity of the yeast was not destroyed while stored in a hydrophobic environment.

### Example 5

Fresh and dry yeast was incorporated into a magnesium stearate/tocopherol (70%/30% mass-related ratio) hydrophobic matrix. The system was stored and shaken in a pH 1 solution (HCl) at body temperature for 90 minutes. After the 90 minutes, saccharose water at body temperature was added to the system. After destruction of the hydrophobic matrix, the yeast showed all signs of yeast survival (carbon dioxide development).

### Example 6

Fresh and dry yeast without a hydrophobic matrix was stored and shaken in a pH 1 solution (HCl) at body temperature for 90 minutes. After the 90 minutes, saccharose water at body temperature was added to the system. The yeast was functionally dead as there was no carbon dioxide development.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms expressed herein.

While the disclosed embodiments have been particularly shown and described with reference to specific embodiments, it should be understood by those skilled in the art that various changes in form and detail may be made therein without departing from the spirit and scope of the disclosed embodiments as defined by the appended claims. The scope of the disclosed embodiments is thus indicated by the appended claims and all changes which come within the meaning and range of equivalency of the claims are therefore intended to be embraced.

What is claimed is:

1. A drug delivery system, comprising:
at least one hydrophobic matrix; and
at least one pharmaceutically active compound.

2. The drug delivery system of claim 1, wherein the hydrophobic matrix comprises at least one hydrophobic solid component and at least one hydrophobic liquid component.

3. The drug delivery system of claim 2, wherein the hydrophobic solid component and the hydrophobic liquid component of the hydrophobic matrix have a stronger binding affinity with each other than with the pharmaceutically active compound.

4. The drug delivery system of claim 2, wherein said hydrophobic solid component comprises an anti-caking agent, said anti-caking agent is a compound selected from the group consisting of magnesium stearate, magnesium palmitate, and similar compounds, and combinations thereof.

5. The drug delivery system of claim 2, wherein the hydrophobic solid component is selected from the group consisting of waxes, fruit wax, carnauba wax, bees wax, waxy alcohols, plant waxes, soybean waxes, synthetic waxes, triglycerides, lipids, long-chain fatty acids and their salts like magnesium stearate, magnesium palmitate, esters of long-chain fatty acids, long-chain alcohols like cetyl palmitate, waxy alcohols, long-chain alcohols like cetylalcohol, oxethylated plant oils, oxethylated fatty alcohols.

6. The drug delivery system of claim 2, wherein the hydrophobic liquid component acts as a glue to bind the hydrophobic solid component together.

7. The drug delivery system of claim 2, wherein the hydrophobic liquid component is selected from the group consisting of plant oils, castor oil, jojoba oil, soybean oil, silicon oils, paraffin oils, and mineral oils, cremophor, oxethylated plant oils, and oxethylated fatty alcohols.

8. The drug delivery system of claim 2, wherein the hydrophobic liquid component is labeled with at least one agent selected from the group consisting of small molecules, hormones, peptides, proteins, phospholipids, polysaccharides, mucins and biocompatible polymers.

9. The drug delivery system of claim 8, wherein the biocompatible polymers comprise polyethylene glycol (PEG), dextran or another similar material.

10. The drug delivery system of claim 1, wherein the drug delivery system comprises a pharmaceutically active compound from about 0.1 mass percent to about 35 mass percent of the drug delivery system, and a hydrophobic matrix, said matrix comprising a hydrophobic solid component from about 60 mass percent to about 95 mass percent of the drug delivery system, and a hydrophobic liquid component from about 40 mass percent to about 5 mass percent of the drug delivery system.

11. The drug delivery system of claim 1, wherein the pharmaceutically active compound is either alone or in combination with at least one excipient.

12. The drug delivery system of claim 11, wherein the excipient is selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof.

13. The drug delivery system of claim 1, wherein the pharmaceutically active compound is selected from the group consisting of a living organelle, a cell, a tissue constituent, a protein, a humanized monoclonal antibody, a human monoclonal antibody, a chimeric antibody, an immunoglobulin, fragment, derivative or fraction thereof, a synthetic, semi-synthetic or biosynthetic substance mimicking immunoglobulins or fractions thereof, an antigen binding protein or fragment thereof, a fusion protein or peptide or fragment thereof, a receptor antagonist, an antiangiogenic compound, an intracellular signaling inhibitor, a peptide with a molecular mass equal to or higher than 3 kDa, a ribonucleic acid (RNA), a deoxyribonucleic acid (DNA), a plasmid, a peptide nucleic acid (PNA), a steroid, a corticosteroid, an adrenocorticostatic, an antibiotic, an antidepressant, an antimycotic, a [beta]-adrenolytic, an androgen or antiandrogen, an antianemic, an anabolic, an anesthetic, an analeptic, an antiallergic, an antiarrhythmic, an antiarterosclerotic, an antibiotic, an antifibrinolytic, an anticonvulsive, an anti-inflammatory drug, an anticholinergic, an antihistamine, an antihypertensive, an antihypotensive, an anticoagulant, an antiseptic, an antihemorrhagic, an antimyasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a hemostatic, a hormone and its synthetic or biosynthetic analogue, an immunosuppressant, an immunostimulant, a mitogen, a physiological or pharmacological inhibitor of mitogens, a mineralocorticoid, a muscle relaxant, a narcotic, a neurotransmitter, a precursor of neurotransmitter, an oligonucleotide, a peptide, a (para)-sympathomimetic, a (para)-sympatholytic, a sedating agent, a spasmolytic, a vasoconstrictor, a vasodilator, a vector, a virus, a virus-like particle, a virustatic, a wound healing substance and a combination thereof.

14. The drug delivery system of claim 13, wherein the living organelle, the cell or the tissue constituent has a cell wall, wherein said cell wall protects the bioactivity of the pharmaceutically active compound from hydrophobic properties of the hydrophobic matrix.

15. The drug delivery system of claim 1, wherein the hydrophobic matrix and the pharmaceutically active compound are in a paste-like or a semi-solid form.

16. The drug delivery system of claim 1, wherein the pharmaceutically active compound is dispersed in the hydrophobic matrix in a particulate form, in a microparticulate form, or in a dissolved state.

17. The drug delivery system of claim 1, wherein the pharmaceutically active compound is dissolved in an aqueous solution.

18. (canceled)

19. The drug delivery system of claim 1, wherein the hydrophobic matrix comprises an aqueous solution.

20. (canceled)

21. The drug delivery system of claim 1, wherein the hydrophobic matrix is labeled with at least one agent selected from the group consisting of dyes, fluorophores, chemiluminescent agent, isotopes, metal atoms or clusters, radionuclides, enzymes, antibodies and tight binding partners, said tight binding partners comprising biotin or avidin.

22. The drug delivery system of claim 1, wherein said hydrophobic matrix conserves activity of said pharmaceutically active compound in hydrophobic environment, protects functionality of the pharmaceutically active compound in a hostile condition, hydrophobic environment or a combination thereof, provides stability to the pharmaceutically active compound at ambient or elevated temperatures, protects the pharmaceutically active compound from water-soluble poisonous substances, biological attack or a combination thereof, provides a temporary replacement for a cold chain, maintains bioactivity of the pharmaceutically active compound at higher temperature or a combination thereof.

23-29. (canceled)
